(19) United States
(12) Patent Application Publication
(10) Pub. No.: US 2014/0238940 A1
(43) Pub. Date: Aug. 28, 2014

(54) Single Stage Filtration System and Method For Use with Blood Processing Systems

(71) Applicant: Haemonetics Corporation, Braintree, MA (US)

(72) Inventors: Donald J. Schwarz, Bridgewater, MA (US); Steve Mastroyin, Pawtucket, RI (US); Seth Kasper, Cambridge, MA (US)

(73) Assignee: Haemonetics Corporation, Braintree, MA (US)

(21) Appl. No.: 14/272,080

(22) Filed: May 7, 2014

Related U.S. Application Data
(63) Continuation of application No. PCT/US2011/061709, filed on Nov. 21, 2011.

(51) Int. Cl. B01D 65/00; A61M 1/16

(52) U.S. Cl. CPC B01D 65/003; A61M 1/1631
USPC 210/650; 210/257.2; 210/86

(57) ABSTRACT

A reservoir for use with a blood collection system includes a housing defining a cavity and a single stage filter. The housing has an inlet for receiving fluid from a source and an outlet. The inlet is in fluid communication with the cavity. The single stage filter includes a filter membrane configured to filter the fluid entering the housing from the inlet, and a frame defining the structure of the single stage filter. The frame also supports the filter membrane within the housing, and has a wiper edge that seals against an inner wall of the housing.

### PRIORITY

This application is a continuation of co-pending Patent Cooperation Treaty application PCT/US2011/061709, entitled “Single Stage Filtration System and Method For Use with Blood Processing Systems,” filed Nov. 21, 2011, assigned attorney docket number 1611/A72WO, and naming Donald J. Schwarz, Steve Mastroyin, and Seth Kasper as inventors, the disclosure of which is incorporated herein, in its entirety, by reference.

### TECHNICAL FIELD

The present invention relates to methods and systems for the filtration of blood and blood products, and more particularly to single stage filtering of blood entering blood processing equipment and storage devices.

### BACKGROUND ART

It is well known that patients undergoing surgery lose blood both during and after surgery. To compensate for this blood loss, physicians and medical practitioners must replenish the volume of blood lost by the patient and may do so in variety of way. One such known method is to give the patient a blood transfusion with allogenic blood. However, allogenic blood is expensive and the transfusion puts the patient at risk for infection and complications.

To avoid the use of allogenic blood, physicians and medical practitioners often use blood salvage and processing systems. These blood salvage and processing systems allow the physician and/or medical practitioner to collect the patient's own blood, process (e.g., wash) the blood, and autotransfuse the patient with their own blood or blood components. Autotransfusions with the patient's own blood greatly reduce the risk of infection and complications to the patient.

As mentioned above, blood loss not only occurs during surgery, but also post-operative. Accordingly, physicians and medical practitioners often utilize a wound drain to drain the blood from the surgical site. This wound drain may, in turn, be connected to a blood salvage and processing system in order to salvage the blood lost postoperatively.

As one may expect, the blood and fluid removed via the wound drain may contain various particulates such as debris and blood clots. To prevent these particulates from entering the blood processing system and interfering with the system's performance, current systems use filters located between the wound and the blood processing system to remove the particulates. However, the time required to filter incoming blood and the large quantity of hold-up within the prior art filters prevents users/technicians from receiving fast and accurate information regarding the amount of blood collected and lost by the patient.

### SUMMARY OF THE INVENTION

In a first embodiment of the invention there is provided a reservoir for use with a blood collection system. The reservoir includes a housing defining a cavity and a single stage filter. The housing has (1) an inlet in fluid communication with the cavity and for receiving fluid from a source, and (2) an outlet. The single stage filter is located within the cavity, and includes a filter membrane and a frame. The filter membrane is configured to filter the fluid entering the housing from the inlet. The frame defines the structure of the single stage filter and supports the filter membrane within the housing. The frame has a wiper edge that seals against an inner wall of the housing, and may be made from a black medical grade plastic, for example, polypropylene.

The single stage filter may be positioned horizontally within the cavity and may be a mesh screen with a hydrophilic coating (e.g., a plasma coating or a salt based coating that reduces the surface tension between the filter membrane and the fluid) or may be made from a hydrophilic material. The wiper edge may be polypropylene and may extend around the outer periphery of the frame. The frame may include a plurality of support structures that support the filter membrane within the frame.

In accordance with some embodiments of the present invention, the reservoir may also include (1) a dip tube that extends from the outlet of the reservoir to the bottom of the reservoir, and/or (2) a fluid level indicator. The frame may include a flange extending inwardly from an edge of the frame. The flange may have an aperture there through for receiving the dip tube. The aperture, in turn, may include a sealing ring that seals against the dip tube to prevent fluid from passing through the aperture. The fluid level indicator may have a float tube and a float. The float tube may be in fluid communication with the cavity such that the float can rise and drop with the fluid level within the reservoir.

In accordance with further embodiments, a single stage filter for use in a blood collection system reservoir may include a filter membrane and a frame. The filter membrane may be configured to filter fluid entering the housing from a housing inlet. The frame may define the structure of the single stage filter and may be configured to support the single stage filter and filter membrane within a cavity of the blood collection system reservoir. The frame may be made from a black medical grade plastic, for example, polypropylene.

The frame may have a wiper edge that seals against an inner wall of the housing and secures the frame within the housing. The single stage filter may be positioned horizontally within the cavity. The wiper edge may also be polypropylene, and may extend around the outer periphery of the frame. To support the filter membrane within the frame, the frame may include a plurality of support structures.

The filter membrane may be a mesh screen and may have a hydrophilic coating and/or be made from a hydrophilic material. For example, the hydrophilic coating may be a plasma coating. Additionally or alternatively, the filter membrane may have a salt based coating that reduces the surface tension between the filter membrane and the fluid being filtered.

The reservoir may include a dip tube extending from the outlet of the reservoir to the bottom of the reservoir. To allow the dip tube to pass through the single stage filter, the frame may include a flange extending inwardly from an edge of the frame. The flange may have an aperture there through for receiving the dip tube. Additionally, the aperture may include a sealing ring that seals against the dip tube to prevent fluid from passing through the aperture.

In accordance with further embodiments of the present invention, a method for filtering blood using a blood processing system includes (1) connecting a reservoir to the blood collection and processing system, (2) introducing blood into the reservoir through an inlet, and (3) filtering the blood introduced into the reservoir using a single stage filter. The single stage filter may be located within the cavity of the reservoir and may be in fluid communication with the inlet. The filter may include a frame that defines the structure of the single stage filter, and configured to support the single stage filter and filter membrane within the cavity. The frame may also have a wiper edge that seals against an inner wall of the housing and secures the frame within the housing.

The single stage filter may be positioned horizontally within the cavity, and the filter membrane may be a mesh screen with a plasma or salt based coating (or other hydrophilic coating) that reduces the surface tension between the filter membrane and the fluid being filtered. The frame and the wiper edge may be made from black medical grade plastic, for example, polypropylene. The wiper edge may extend around an outer periphery of the frame, and the frame may include a plurality of support structures that support the filter membrane within the frame.

The method may also include extracting filtered blood from the reservoir via an outlet in fluid communication with the cavity, and introducing the extracted blood into a blood processing device. Extracting the filtered blood may include drawing the filtered blood through a dip tube extending from the outlet of the reservoir to the bottom of the reservoir. The filter frame may include a flange extending inwardly from an edge of the frame and having an aperture there through for receiving the dip tube. The aperture may include a sealing ring that seals against the dip tube to prevent fluid from passing through the aperture.

### BRIEF DESCRIPTION OF THE DRAWINGS

The foregoing features of the invention will be more readily understood by reference to the following detailed description, taken with reference to the accompanying drawings, in which:

FIG. 1 is an isometric view of a blood processing device and reservoir, in accordance with some embodiments of the present invention.

FIG. 2 schematically shows an isometric view of an alternative embodiment of a reservoir with the reservoir wall transparent to show the internal cavity of the reservoir, in accordance with some embodiments of the present invention.

FIG. 3 schematically shows an exploded view of the reservoir shown in FIG. 2, in accordance with various embodiments of the present invention.

FIGS. 4A-E schematically show various views and details of a single stage filter used within the reservoir shown in FIG. 3, in accordance with some embodiments of the present invention.

FIG. 5 schematically shows a back view of the reservoir shown in FIG. 2, in accordance with various embodiments of the present invention.

FIG. 6 is a flowchart showing a method of using the reservoir shown in FIG. 2 to pre-filter and filter blood, in accordance with some embodiments of the present invention.

### DETAILED DESCRIPTION OF SPECIFIC EMBODIMENTS

In illustrative embodiments, a reservoir-and-filter system may be used in conjunction with blood processing systems and devices to allow physicians and medical practitioners to process a patient's own blood and return processed blood (or individual blood components) back to the patent. Additionally, some embodiments of the present invention may include a single stage filter that improves filtration efficiency.

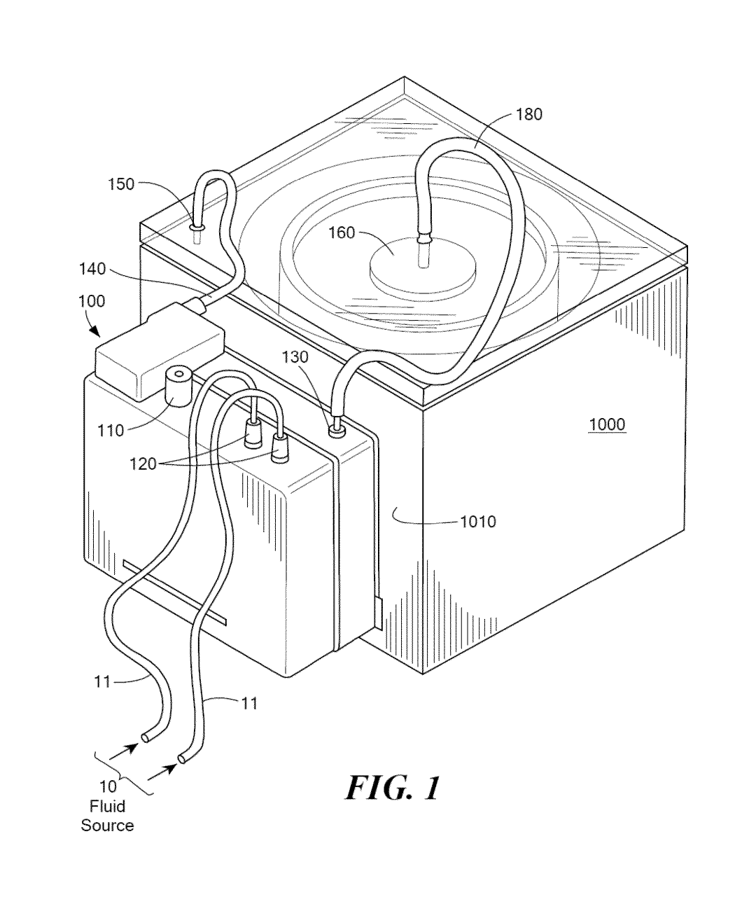

FIG. 1 shows a reservoir 100 and a blood processing system 1000 in accordance with embodiments of the present invention. The reservoir 100 may be connected to a side 1010 of the blood processing device 1000. Tubing and various inlets and outlets may facilitate the transfer of fluids (e.g., blood and blood components) in and out of the reservoir 100 and blood processing device 1000. For example, unfiltered fluid obtained from a fluid source 10 (e.g., a wound drain, a blood storage container, an intra-operative surgical site, etc.) may be directed into the reservoir 100 through inlets 110, 120 (e.g., through tubes 11). It is important to note that the inlet used to introduce the fluid into the reservoir 100 may be dependent upon the application. For example, blood introduced intra-operatively may enter through inlets 120, whereas blood introduced post-operatively (e.g., from a wound drain) may enter through inlet 110. Moreover, because fluid introduced from a wound drain may contain sizeable particulates, inlet 110 may have a larger inner diameter to accommodate the particulates. Fluid may be removed from the reservoir 100 (e.g., for processing within blood processing device 1000) via the outlet 130. This outlet 130 may be fluidly connected to the blood processing device 1000 (and, in particular, a separation device 160) via fluid tube 180.

As mentioned above, fluids such as blood and blood products may enter and exit the reservoir 100. To that end, the reservoir 100 may be connected to a vacuum source 150 via vacuum line 140. The vacuum source 150 may be used to create vacuum and pressure differentials within the reservoir 100 and/or blood processing device 1000 to aid in the transfer of fluids in and out of the various components of the system.

As also mentioned above, the reservoir 100 and blood processing device 1000 may be used for a variety of applications (e.g., intra-operative, postoperative, etc.). For ease of understanding, illustrative embodiments described herein will be discussed in reference to a wound-drain application. However, it is important to note that the reservoir 100 and blood processing device 1000 described herein can be used for a variety of other applications including, but not limited to, intra-operative applications, other post-operative application, or processing previously collected and stored blood or blood products (e.g., blood and blood products that are stored in blood bags/containers).

In the wound drain application mentioned above, the fluid source 10 may be or may be in fluid communication with a post-operative surgical site where blood, clots, debris, and other fluids are present and/or generated. Prior to processing the fluid emanating from the wound site and/or returning some or all of the components back to the patient, it is important to remove debris and clots from the blood/fluid because such debris and clots may be problematic during processing, hazardous if returned to the patient, and undesirable if the collected blood/fluid is to be stored for later use. To that end, some embodiments of the present invention have various components within the reservoir 100 that pre-filter and filter the fluid entering through the inlet 110. These pre-filtration and filtration components are described in greater detail below.

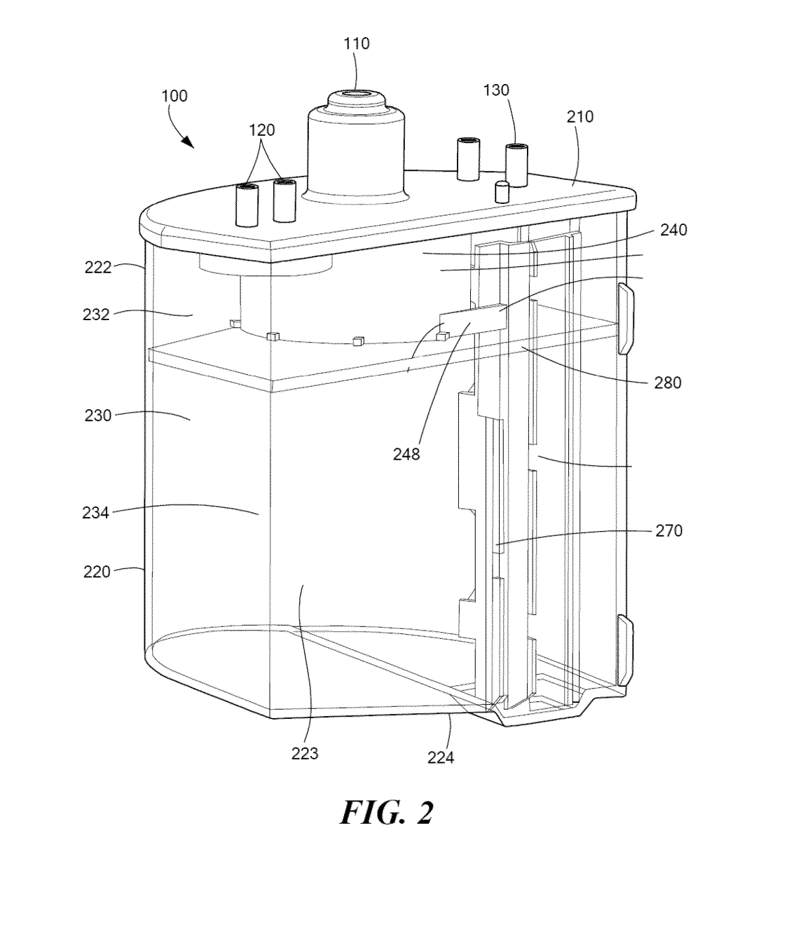

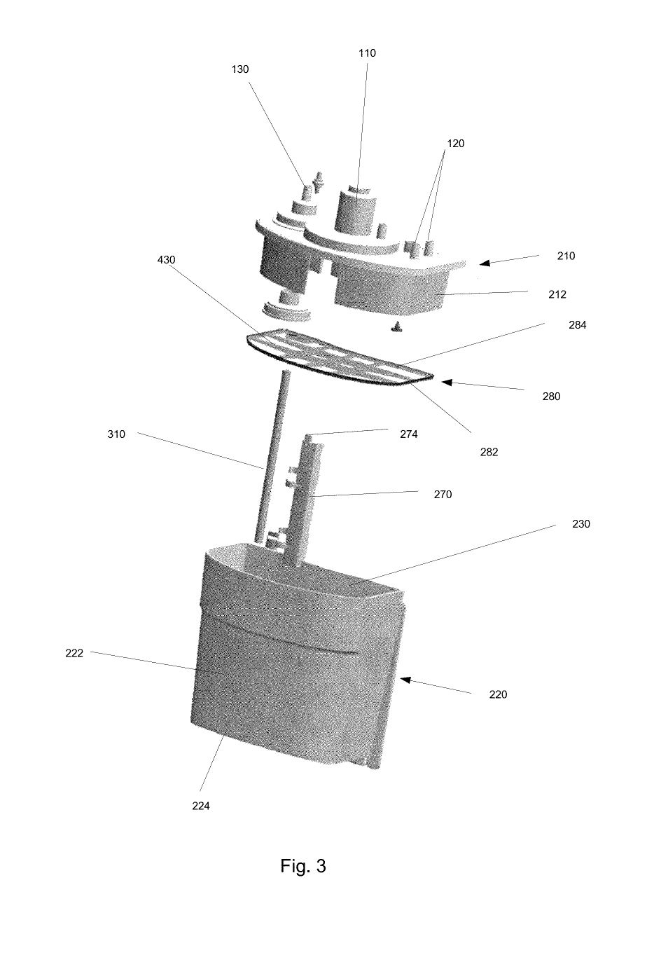

FIG. 2 shows an alternative embodiment of the reservoir 100 with a transparent wall in order to show the internal cavity of the reservoir 100. FIG. 3 shows an exploded view of the reservoir 100 to show the internal components. As shown in FIGS. 2 and 3, the reservoir 100 may have a cover 210 with a skirt 212 (FIG. 3), and a housing 220 forming an internal cavity 230. The inlets 110/120 and the outlet 130 (described above) may be located within the cover 210. In order to increase structural strength and rigidity, the housing 220 may have at least one curved wall 222. For example, as shown in FIGS. 2 and 3, the housing 220 may be D-shaped. If additional strength or rigidity is needed, the housing 220 may have ribs located on the walls.

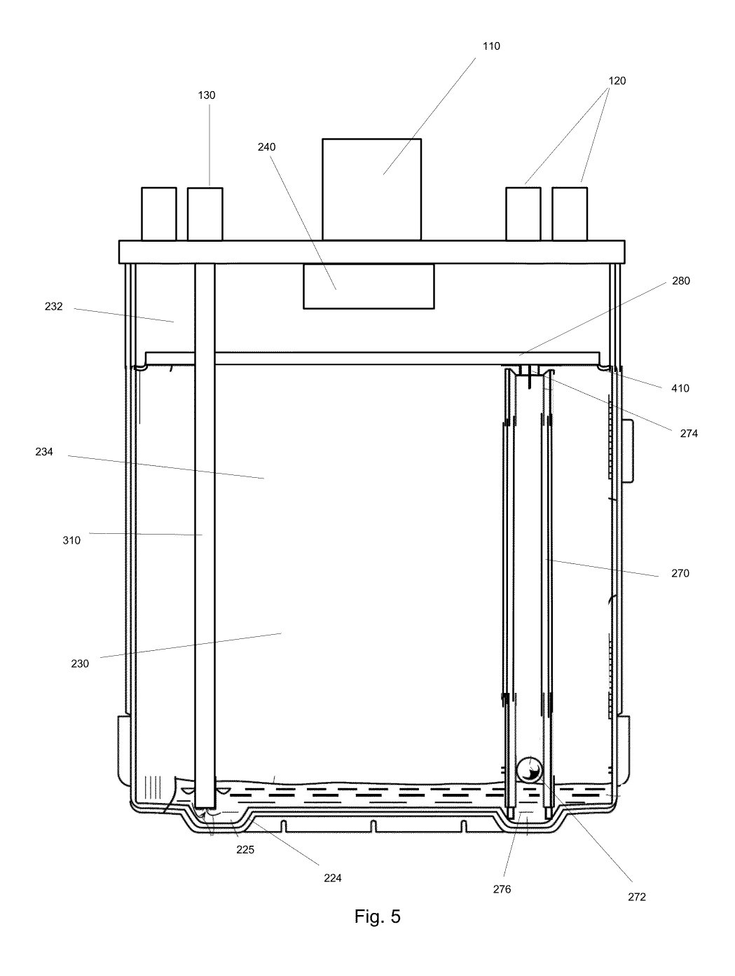

As discussed in greater detail below, filtered blood is collected at the bottom of the reservoir 100. Accordingly, the outlet 130 may be fluidly connected to a dip-tube 310 (FIG. 3) extending from the outlet 130 to the bottom of the housing 220. In order to allow the maximum amount of blood/fluid to be extracted from the housing 220, the base 224 of the housing 220 may be angled toward the dip tube 310 and/or the base may have a recess 225 (FIG. 5), ensuring that fluid within the housing 220 will flow towards and gather at the bottom of the dip-tube 310.

In order to filter the blood entering the inlet 120, the reservoir 100 may include a single stage filter 280 located within the cavity 230 and spaced from the inlet 120. As shown in FIGS. 2 and 3, the filter 280 may be oriented horizontally within the cavity 230. In this orientation, the filter 280 essentially divides the reservoir 100 into a pre-filtered portion 232 and a filtrate portion 234. The pre-filtered portion 232 contains fluid/blood that has entered the reservoir but has not yet been filtered. The filtrate portion 234 contains/holds any fluid/blood that has already been filtered and is awaiting extraction (discussed in greater detail below).

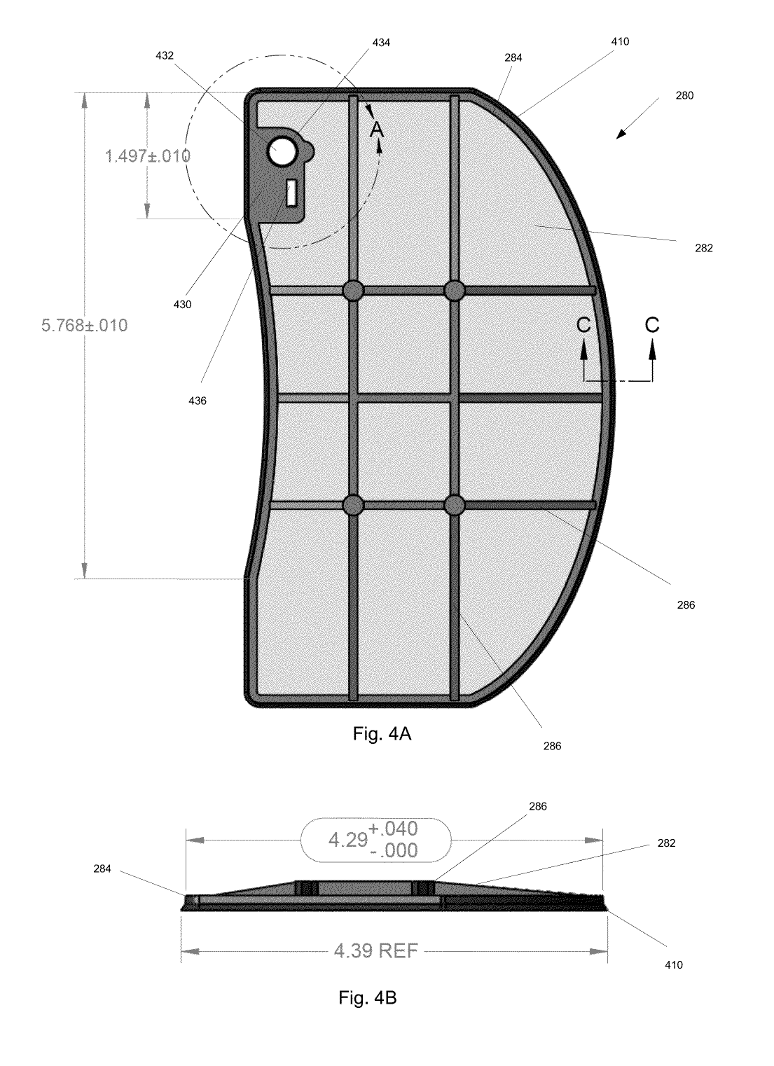

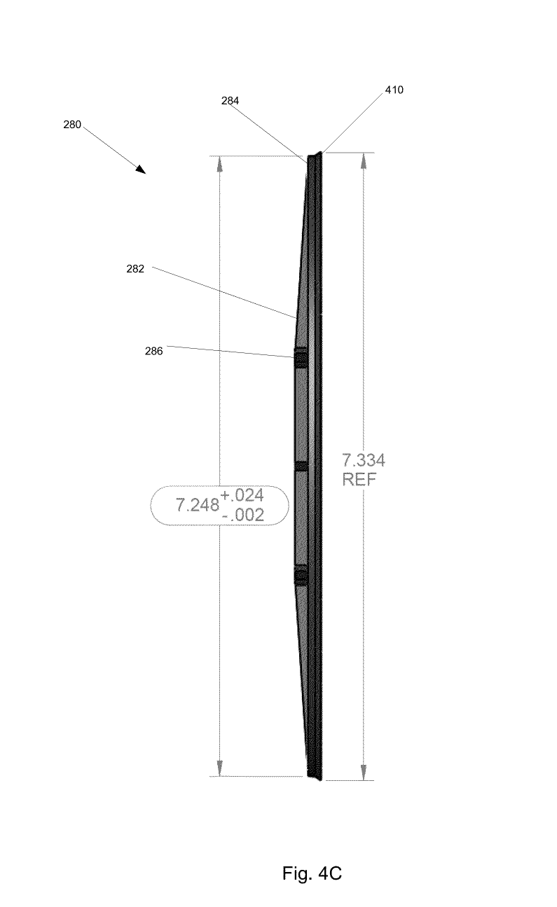

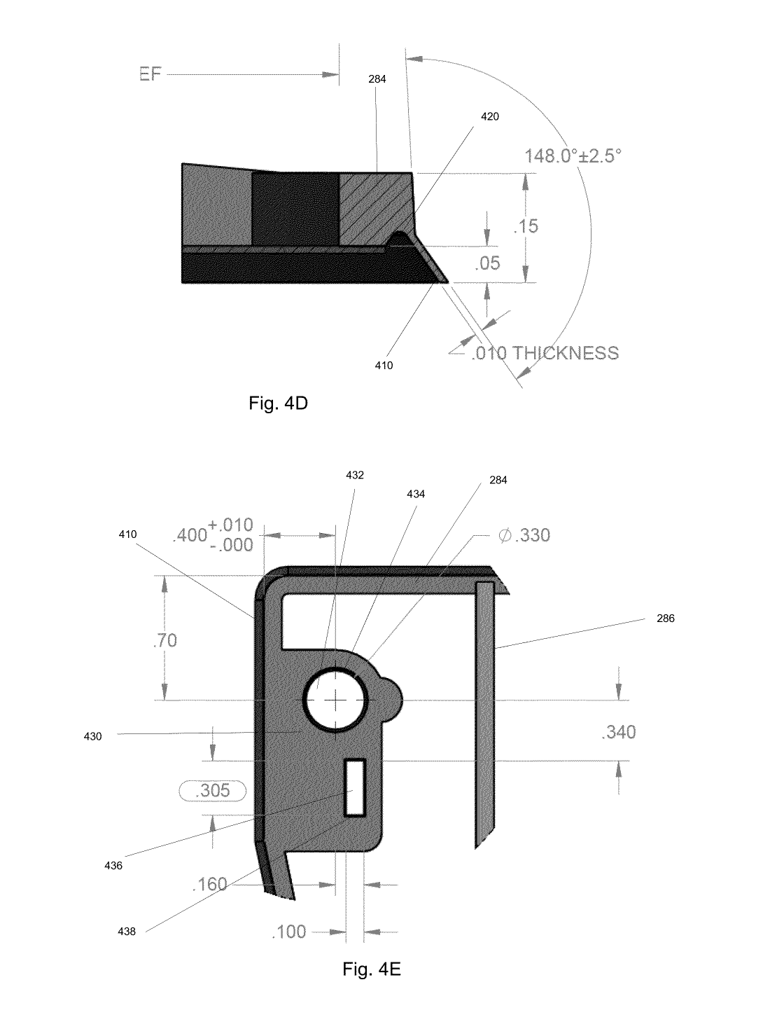

As best shown in FIGS. 4A-4C, the single stage filter 280 may include a filter membrane 282 and a frame 284. The frame 284 defines the structure of the single stage filter 280 and may, in turn, include a number of support members 286 that provide additional strength and rigidity to the frame 284. Additionally, the support members 286 act to support the filter membrane 282 during filtration and prevent the filter membrane 282 from sagging/deforming under the weight of the fluid entering the reservoir 100 and any material that is filtered out of the blood and collected on the filter 280. For additional filter support, as discussed above and as shown in FIG. 3, some embodiments of the cover 210 may also include a skirt 212 (FIG. 3) that supports the filter 280 from above and prevents deflection and/or deformation of the filter 280 towards the inlet 110 when the reservoir 100 is assembled (e.g., as shown in FIGS. 2 and 5).

As best shown in FIG. 4D, the frame 284 may include a wiper edge 410 that extends slightly outwards and downwards from the frame 284. The wiper edge 410 may extend around the entire periphery of the frame 284 and create a seal against the inner wall of the reservoir 100 when the filter 280 is installed. For example, the frame 284 of the single stage filter 280 and the wiper edge 410 may be sized to be slightly larger than the inner dimensions of the reservoir 100 (e.g., slightly larger than the dimensions of the cavity 230). Therefore, as the single stage filter 280 is inserted into the cavity 230, the inner wall of the reservoir 100 will cause the wiper edge 410 to deform (e.g., the wiper edge 410 will flex/deform about point 420) and press against the inner wall. As the wiper edge 410 continues to deform and press against the inner wall, the wiper edge 410 creates a seal against the inner wall to prevent the unfiltered blood from leaking by the outer periphery of the single stage filter 280 and into the filtrate portion 234.

It is important to note that various embodiments of the present invention do not require additional supports, securing mechanisms, or adhesives to hold the filter 280 in place within the reservoir 100. Rather, the forces created between the wiper edge 410 and the inner wall of the reservoir 100 as the filter 280 is installed are sufficient to secure/hold the filter 280 in place during shipping and normal operation. To that end, the wiper edge 410 may essentially allow the filter 280 to be press-fit into the reservoir 100/cavity 230.

The frame 284 of the single stage filter 280 may be made from any number of materials that provides sufficient rigidity to support the filter membrane 282, yet remain flexible enough to allow the wiper edge 410 to deform and seal against the inner wall of the reservoir 100. For example, in some embodiments, the frame 284 may be made from polypropylene and may be black in color. As discussed in greater detail below, in embodiments in which the frame 284 is black in color, the single stage filter 280 may be used as a line of demarcation during calibration of the optical sensor.

Although FIG. 4D shows the wiper edge 410 being integrally formed with the frame 284 of the single stage filter 280 and, thus, made from the same material, it is important to note that in some embodiments, the wiper edge 410 may be a separate element. For example, in some embodiments, that wiper edge 410 may be made from a different material (e.g., a more flexible/resilient material) and may be secured or bonded to the frame 284. In such embodiments, the wiper edge 410 may be glued, ultrasonically welded, overmolded, or otherwise chemically or physically bonded/secured to the frame 284.

In some embodiments, the filter membrane 282 can be a fine mesh screen. For example, the filter membrane 282 can be a polyester mesh screen having a mesh opening of approximately 200 microns. An exemplary mesh screen that may be used by various embodiments of the present invention is manufactured by SAATItech™—in particular Saatifil® PES200/43. However, the size and type of the filter membrane may be adjusted depending on the application. For example, screens with a larger or smaller mesh opening may be used depending on the application.

Additionally, the filter membrane 282 may be treated or coated to improve filtration time and efficiency, and reduce filter hold-up (e.g., the amount of fluid that sits on top of the filter during filtration). For example, filter membrane 282 may be plasma treated/coated. As is known in the art, plasma treatment/coating essentially roughens the surface of the mesh (e.g., as the noble gases used during the treatment process bond with the filter membrane). This increase in surface roughness creates a hydrophilic plasma coating that improves filtration efficiency and strike through, and reduces the amount of hold-up on the filter membrane 282 (e.g., the plasma coating allows the fluid to pass through the filter membrane 282 faster and reduces the amount of fluid that collects on top of the filter prior to filtration).

Additionally or alternatively, the filter membrane 282 may have a salt based coating. In a manner similar to the plasma treatment/coating, the salt based coating breaks up the surface tension of the filter membrane to improve the filtration efficiency and strike through.

As mentioned above, the reservoir 100 may have a dip tube 310 through which filtered fluid/blood may be extracted. To that end, the filter 280 may have a flange 430 extending inwardly from the frame 284. The flange 430 may have a through hole 432 (e.g., and aperture) that allows the dip tube 310 to pass through the filter 280 (e.g., so that the dip tube 310 can extend from the outlet 130 to the bottom of the reservoir 100). Like the outer periphery of the frame 284, the inner diameter of the through hole 432 may also have a wiper edge 434 that creates a seal against the dip tube 310 when inserted through the hole 432 (e.g., to prevent fluid from leaking through the hole 432). Alternatively, the hole 432 may have an o-ring or other sealing member that seals against the dip tube 310.

In addition to the hole 432 for the dip tube 310, the flange 430 may also include a notch 436. As discussed in greater detail below the notch 436 aids in securing a float tube 270 within the reservoir 100. In a manner similar to the hole 432, the notch 436 may also have a wiper edge 438 or other sealing member (e.g., an o-ring) that seals against a tab 274 on the float tube 270 (FIG. 5).

As shown in FIGS. 2 and 5, the reservoir 100 may include a float tube 270 extending vertically along the inner wall of the reservoir 100. The upper end of the float tube 270 may include a tab 274 that mates with the notch 436 within the flange 430 of the frame 284. A float 272 may be positioned within the float tube 270 and may be free to move up and down within the float tube 270 such that, as the fluid level within the reservoir 100 rises, the float 272 will also rise. To that end, the float 272 and the portion of the float tube 270 located within the filtrate portion 234 may be used to determine the amount of filtered fluid contained within the reservoir 100. For example, the bottom 276 of the float tube 270 may be open such that filtered fluid within the reservoir 100 will enter the bottom portion of the float tube 270 causing the float 272 to rise with the fluid level. An optical sensor within the blood processing device 1000 may then be used determine the fluid level within the reservoir 100 based on the height at which the float 272 sits.

The float tube 270 may be located next to and on the same side of the reservoir 100 as the dip tube 310, or it may be located elsewhere within the reservoir 100. For example, as shown in FIGS. 2 and 3, the float tube 270 may be located adjacent to the dip tube 310, and the flange 430 may include both the hole 432 for the dip tube 310 and the notch 436 for the tab 274. Alternatively, as shown in FIG. 5, the float tube 270 may be spaced apart from the dip tube 310 (e.g., it may be located on the other side of the reservoir 100). In such embodiments, the filter 280 may have a second flange (not shown) that includes the notch 274 or the flange 430 may extend across the width of the filter 280 such that the hole 432 is at one end of the flange, and the notch 436 is at the other end of the flange 430.

As mention above, in embodiments having a filter frame 284 made from black polypropylene (or other black medical grade plastic), the filter frame 284 may be used a line of demarcation during calibration of the optical sensor. For example, the filter frame 284 may act as a “full line” during calibration. In other words, during calibration, the optical sensor can use the bottom of the reservoir 100 (or the location of the float 272 when the reservoir 100 is empty) as the zero point (e.g., the point indicating that the reservoir 100 is empty) and the filter frame 284 as the full line. The blood processing device 1000 may then determine the amount of fluid within the reservoir 100 (or the amount of space remaining within the reservoir 100) based upon the height of the float 272.

It is important to note that the various embodiments of the present invention may also be used in conjunction with the pre-filter and integrated measurement system described in U.S. application Ser. No. 12/564,514 (filed on Sep. 22, 2009, published as US Publication No. 2011/0068061, and incorporated herein by reference). For example, as shown in FIG. 2, some embodiments of the present invention may have a pre-filter 240 located within the cavity 230 of the housing. The pre-filter 240 may be located just downstream of and in fluid communication with the inlet 110.

The pre-filter 240 located within the reservoir cavity 230 may include a spring mechanism (not shown) that allows the pre-filter 240 to travel within the cavity 230 of the reservoir 100. For example, as the pre-filter 240 removes the debris and clots from the fluid entering the reservoir 100, the debris and clots begin to weigh down the pre-filter 240. The spring mechanism, in turn, will allow the pre-filter 240 to travel downward as the volume (and, thus the weight) of the collected debris/clots/particulates increases within the pre-filter 240. The blood processing device 1000 may measure the amount of debris/clots collected within the pre-filter 240 by determining the location of a location arm 248 that moves up and down with the pre-filter (e.g., by using the same or similar optical sensor used to determine the position of the float 272 within the float tube 270).

It is important to note that various embodiments of the present invention provide numerous benefits over prior art reservoir and filtration systems. In particular, by reducing the amount of hold-up and fluid that sits on top of the filter membrane 282 prior to being filtered, embodiments of the present invention are able to provide users and technicians with a more accurate measure of blood loss. For example, by monitoring the amount of filtered fluid/blood below the filter (e.g., fluid/blood that has been filtered), a user/technician will be able to know sooner if the patient is loosing too much blood. Additionally, if the blood processing system 1000 is similar to the blood processing system described in U.S. patent application Ser. No. 11/936,595 (filed on Nov. 7, 2007, published as U.S. Publication No. 2008/010893, and incorporated herein by reference) and allows the user/technician to delay the installation of a separation device 160 until a predetermined amount of blood is collected within the reservoir, the user/technician will have more accurate information sooner so that she/he may decide whether or not to install the separation device 160 into the processing system 1000.

Furthermore, in applications in which the flow of blood into the reservoir 100 is low (e.g., the blood merely trickles into the reservoir 100), the blood will be filtered as it enters the reservoir 100 (e.g., there will be minimal filter hold-up). Conversely, in prior art systems with greater filter hold-up, a large quantity of blood needs to collect on the filter before filtration starts. As one may expect, this delay increases processing/filtration time and prevents technicians from receiving fast and accurate information regarding blood loss.

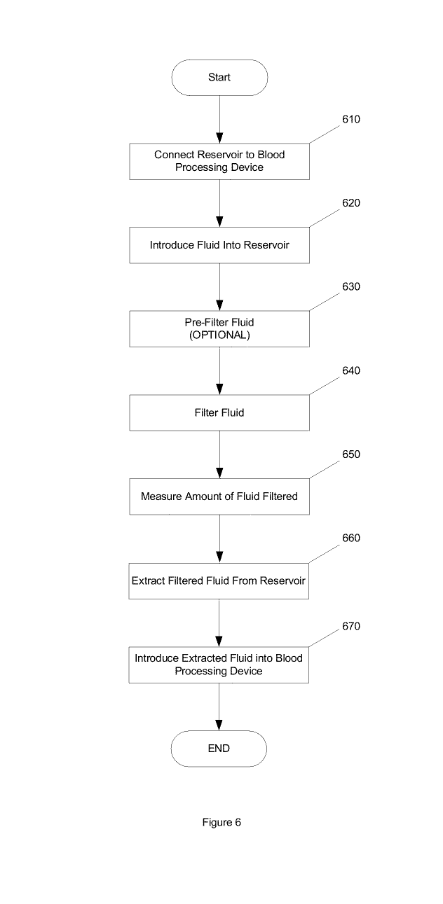

FIG. 6 schematically shows a flowchart depicting a method of using the reservoir 100 and blood processing device 1000 described above. In particular, a physician or medical practitioner may connect the reservoir 100 to the blood processing device 1000 (Step 610). For example, the physician/medical practitioner may first connect the outlet 130 to the blood processing device 1000 using fluid tube 180, connect any required vacuum sources 150 or tubing 140, and orient the reservoir 100 such the flat wall 223 is adjacent the blood processing device and the optical sensor is capable of viewing the float tube 270 and float 272. Additionally, once the reservoir 100 is in place, the medical practitioner may calibrate the optical sensor using the float 272 and singe stage filter 280 as described above.

Once the reservoir 100 is connected, the physician/medical practitioner may then connect the fluid source 10 (e.g., the wound drain or fluid container) to the inlet 110 and begin introducing the fluid into the reservoir 100 (Step 620). If the reservoir 100 is equipped with the pre-filter 240 and integrated measurement system described above, the pre-filter 240 will remove the debris, clots, and particulates from the fluid (Step 630). As the debris, clots, and particulates begin to collect within the pre-filter 240, the weight of the pre-filter 240 will begin to compress the spring mechanism 250 and the pre-filter will travel downward within the cavity 230. As described in U.S. application Ser. No. 12/564,514 (filed on Sep. 22, 2009, published as US Publication No. 2011/0068061, and incorporated herein by reference), the optical sensor may then detect the distance that the pre-filter 240 travels, and the blood processing system 1000 may calculate the volume of particulates removed from the fluid. The blood processing system, different system, or the physician/medical practitioner may then, in turn, use the calculated volume to calculate the estimated fluid loss.

After the incoming fluid has been pre-filtered, the fluid may then pass through the single stage filter 280 (Step 640) and may be collected in the bottom portion of the reservoir 100 (e.g., the filtrate portion 234). As the fluid is filtered and the fluid level within the filtrate portion 234 increased, the optical sensor can measure the amount of fluid that has been collected within the reservoir (Step 650). Once within the filtrate portion 234 of the reservoir, the method may then, optionally, extract the filtered fluid from the reservoir 100 using the dip-tube and the outlet 130 (Step 660) and introduce the removed fluid into the blood processing device 1000 (Step 670) for further processing and/or return to the patient.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A reservoir for use with a blood collection system comprising:
a housing defining a cavity, the housing having an inlet for receiving fluid from a source, the inlet being in fluid communication with the cavity, the housing also having an outlet; and
a single stage filter located within the cavity, the single stage filter including:
a filter membrane configured to filter the fluid entering the housing from the inlet, and
a frame defining the structure of the single stage filter and supporting the filter membrane within the housing, the frame having a wiper edge that seals against an inner wall of the housing.

2. A reservoir according to claim 1, wherein the single stage filter is positioned horizontally within the cavity.

3. A reservoir according to claim 1, wherein the filter membrane is a mesh screen.

4. A reservoir according to claim 1, wherein the mesh screen includes a hydrophilic coating.

5. A reservoir according to claim 4, wherein the hydrophilic coating is a plasma coating.

6. A reservoir according to claim 1, wherein the filter membrane includes a salt based coating, the salt based coating reducing the surface tension between the filter membrane and the fluid.

7. A reservoir according to claim 1, wherein the wiper edge is polypropylene.

8. A reservoir according to claim 1, wherein the wiper edge extends around an outer periphery of the frame.

9. A reservoir according to claim 1, wherein the frame includes a plurality of support structures, the support structures supporting the filter membrane within the frame.

10. A reservoir according to claim 1 further comprising a dip tube extending from the outlet of the reservoir to the bottom of the reservoir.

11. A reservoir according to claim 10 wherein the frame includes a flange extending inwardly from an edge of the frame, the flange having an aperture therethrough for receiving the dip tube.

12. A reservoir according to claim 11, wherein the aperture includes a sealing ring that seals against the dip tube to prevent fluid from passing through the aperture.

13. A reservoir according to claim 1, wherein the frame is made from a black medical grade plastic.

14. A reservoir according to claim 13, wherein the black medical grade plastic is polypropylene.

15. A reservoir according to claim 1 further comprising a fluid level indicator having a float tube and a float, the float tube being in fluid communication with the cavity such that the float can rise and drop with a fluid level within the reservoir.

16. A single stage filter for use in a blood collection system reservoir comprising:
a filter membrane configured to filter fluid entering the housing from a housing inlet; and
a frame defining the structure of the single stage filter and configured to support the single stage filter and filter membrane within a cavity of the blood collection system reservoir, the frame having a wiper edge that seals against an inner wall of the housing and secures the frame within the housing.

17. A single stage filter according to claim 16, wherein the single stage filter is positioned horizontally within the cavity.

18. A single stage filter according to claim 16, wherein the filter membrane is a screen.

19. A single stage filter according to claim 16, wherein the screen includes a hydrophilic coating.

20. A single stage filter according to claim 19, wherein the hydrophilic coating is a plasma coating.

21. A single stage filter according to claim 16, wherein the filter membrane includes a salt based coating, the salt based coating reducing the surface tension between the filter membrane and the fluid.

22. A single stage filter according to claim 16, wherein the wiper edge is polypropylene.

23. A single stage filter according to claim 16, wherein the wiper edge extends around an outer periphery of the frame.

24. A single stage filter according to claim 16, wherein the frame includes a plurality of support structures, the support structures supporting the filter membrane within the frame.

25. A single stage filter according to claim 16, wherein the reservoir includes a dip tube extending from an outlet of the reservoir to the bottom of the reservoir, the frame including a flange extending inwardly from an edge of the frame, the flange having an aperture therethrough for receiving the dip tube.

26. A single stage filter according to claim 25, wherein the aperture includes a sealing ring that seals against the dip tube to prevent fluid from passing through the aperture.

27. A single stage filter according to claim 16, wherein the frame is made from a black medical grade plastic.

28. A single stage filter according to claim 27, wherein the black medical grade plastic is polypropylene.

29. A method for filtering blood in a blood processing system comprising:
connecting a reservoir to the blood collection and processing system, the reservoir having an inlet for receiving blood from a source and an outlet for removing filtered blood from the reservoir;
introducing blood into the reservoir through the inlet; and
filtering the blood introduced into the reservoir using a single stage filter located within the cavity of the reservoir and in fluid communication with the inlet, the single stage comprising:
a filter membrane configured to filter the fluid entering the housing from the inlet, and
a frame defining the structure of the single stage filter and configured to support the single stage filter and filter membrane within the cavity, the frame having a wiper edge that seals against an inner wall of the housing and secures the frame within the housing.

30. A method according to claim 29, wherein the single stage filter is positioned horizontally within the cavity.

31. A method according to claim 29, wherein the filter membrane is a screen.

32. A method according to claim 31, wherein the screen includes a hydrophilic coating.

33. A method according to claim 32, wherein the hydrophilic coating is a plasma coating.

34. A method according to claim 29, wherein the filter membrane is made from a hydrophilic material.

35. A method according to claim 29, wherein the filter membrane has a salt based coating, the salt based coating reducing the surface tension between the filter membrane and the blood.

36. A method according to claim 29, wherein the wiper edge is polypropylene.

37. A method according to claim 29, wherein the wiper edge extends around an outer periphery of the frame.

38. A method according to claim 29, wherein the frame includes a plurality of support structures, the support structures supporting the filter membrane within the frame.

39. A method according to claim 29, further comprising:
extracting filtered blood from the reservoir via an outlet in fluid communication with the cavity; and
introducing the extracted blood into a blood processing device.

40. A method according to claim 39, wherein extracting the filtered blood includes drawing the filtered blood through a dip tube extending from the outlet of the reservoir to the bottom of the reservoir, the filter frame including a flange extending inwardly from an edge of the frame and having an aperture therethrough for receiving the dip tube.

41. A method according to claim 40, wherein the aperture includes a sealing ring that seals against the dip tube to prevent fluid from passing through the aperture.

42. A method according to claim 29, wherein the frame is made from a black medical grade plastic.
